(19) United States
(12) Patent Application Publication
(10) Pub. No.: US 2007/0027511 A1
(43) Pub. Date: Feb. 1, 2007

(54) Collagen injection combined with focused ultrasound for the treatment of incontinence

(76) Inventor: Ty Fairneny, Marlborough, MA (US)

Correspondence Address: FAY KAPLUN & MARCIN, LLP, 15O BROADWAY, SUITE 702, NEW YORK, NY, 10038, US

(21) Appl. No.: 11/194,238

(22) Filed: Aug. 1, 2005

(51) Int. Cl. A61F 7/00; A61F 7/12

(52) U.S. Cl. 607/96; 607/113

(57) ABSTRACT

A method for treating incontinence, comprises inserting a high energy ultrasound probe into a body canal and transmitting ultrasound energy from the probe through a wall of the body canal to focus at a first treatment region of an adjacent portion of endopelvic fascia, an intensity and duration of the transmitted energy being controlled to heat the first treatment region sufficiently to obtain a desired shrinkage of a portion of the endopelvic fascia including the first treatment region.

### BACKGROUND

Urinary incontinence affects a large number of adult females and stress incontinence is a common type of female incontinence. Stress incontinence can result from, for example, detrusor instability (i.e., involuntary contraction of the detrusor muscle) or an intrinsic sphincter deficiency. These conditions are more prevalent in older women and in women who have undergone surgical procedures for the treatment of stress incontinence where scarring or denervation prevent the urethral sphincter from maintaining a water-tight seal when at rest. One of the most common forms of stress urinary incontinence is genuine stress incontinence. This condition, which is caused by a defect in the endopelvic fascia under the bladder neck, results in urethral hypermobility, or displacement of the urethra and the bladder neck to an abnormal position.

Incontinence has been treated by, for example, pelvic floor muscle exercises, the introduction of periurethral bulking agents (e.g., collagen) to constrict the urethra or the urethral sphincter or a number of surgical options including, for example, various bladder suspension procedures that reduce bladder neck and urethra hypermobility by tightening the endopelvic fascia. In addition, radio-frequency (RF) energy has been used to shrink and stabilize the endopelvic fascia to improve support for the urethra and bladder neck. In these procedures an incision is made through the vagina, lateral to the urethra to expose the endopelvic fascia for the application of RF energy thereto. Although RF procedures are less invasive than surgery, they may still involve complication and/or patient discomfort.

### SUMMARY OF THE INVENTION

In one aspect, the present invention is directed to a method for treating incontinence, comprising inserting a high energy ultrasound probe into a body canal and transmitting ultrasound energy from the probe through a wall of the body canal to focus at a first treatment region of an adjacent portion of endopelvic fascia, an intensity and duration of the transmitted energy being controlled to heat the first treatment region sufficiently to obtain a desired shrinkage of a portion of the endopelvic fascia including the first treatment region.

### BRIEF DESCRIPTION OF THE DRAWING

FIG. 1 is a cross sectional view showing an ultrasound probe in the region surrounding the urethra below the bladder neck, according embodiments of the present invention.

### DETAILED DESCRIPTION

The present invention may be further understood with reference to the following description and to the appended drawings, wherein like elements are referred to with the same reference numerals. The present invention is related to methods and devices for treating incontinence. In particular, the present invention is related to procedures and devices utilizing ultrasound to treat urinary incontinence in women.

The present invention provides techniques for treating genuine urinary stress incontinence (USI) using high intensity ultrasound energy to shrink and stabilize the endopelvic fascia supporting the urethra enabling it to maintain the urethra and the bladder neck in a more desirable position. According to the invention, a vaginally inserted probe treats selected portions of the endopelvic fascia through the vaginal wall. Thus the procedure according to the invention can be carried out without incisions or other invasive surgical methods. A probe for use in conjunction with the methods described in this application is described in more detail in U.S. patent application Ser. No. 11/092,463, filed Mar. 29, 2005 and entitled Apparatus and Method for Stiffening Tissue, the entire disclosure of which is hereby expressly incorporated by reference herein.

The procedure may also include inserting additional collagen to the treatment site, to increase the amount of shrinking that affects the tissue during the procedure. In addition, the added collagen acts as a bulking agent, filling spaces vacated by the tissue of the endopelvic fascia as it shrinks.

When tissue is heated, collagen fibers contained therein shrink as they are denatured, shrinking the tissue itself. More specifically, collagen shrinks through the cumulative effects of the “unwinding” of the triple helix in which collagen molecules are shaped. This is due to the destruction of heat-stable intra-molecular cross links, and the resulting residual tension of the remaining heat-stable intermolecular cross links.

Typically, the collagen denatures at a temperature of about 65° Centigrade (C). A more optimized temperature can be determined by performing tests in which biological tissues are heated, and the resulting shrinking is measured. For example, collagen samples taken from different regions of cadaver glenohumeral joint capsules have been heated and exemplary tests show heating to 65° C. for 10 minutes results in a collagen shrinkage of about 10%. Heating of the tissue at 80° C. for 1.5 minutes results in collagen shrinkage of about 60%. Thus the extent of the shrinkage, as well as the time required to achieve a desired level of shrinkage depends on the temperature at which the tissue is heated. The duration and temperature of the heating can thus be optimized to obtain a desired level of tightening of the tissue.

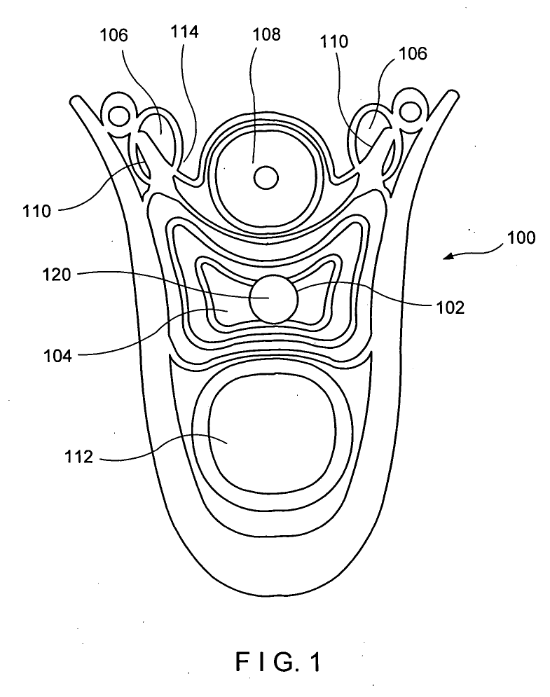

According to exemplary embodiments of the present invention, high intensity ultrasonic energy is used to heat portions of the endopelvic fascia and cause collagen denaturization to add more support to the urethra. By increasing the support of the urethra at the bladder neck, the symptoms of urinary stress incontinence can be greatly reduced. As shown in FIG. 1, the treatment system 100 comprises a high intensity ultrasonic probe 102 sized and shaped for insertion into the vaginal canal 104 to be placed in the vicinity of target portions of the endopelvic fascia 114. The probe 102 is inserted in such a manner that an emitter 120 thereof is located in the vicinity of treatment regions 106 for the delivery through the vaginal wall of ultrasound energy to the treatment regions 106, where the duration and intensity of the ultrasound energy is selected to achieve a desired shrinkage of the target portions of the endopelvic fascia 114.

The treatment regions 106 are preferably located around both sides of the urethra 108, to more evenly tighten the fascia 114. For example, the treatment regions 106 are preferably located substantially symmetrically around the urethra 108. Of course, those skilled in the art will understand that the exact location of the treatment regions 106 will vary from one patient to another, based on the patient's anatomy and the desired amount of tightening of the endopelvic fascia 114. In one exemplary embodiment, the treatment regions 106 are about 1 cm lateral to either side of the urethra 108. The size of the treatment regions 106 also may also be varied to customize the shrinkage of the endopelvic fascia 114. For example, exemplary treatment regions 106 may be about 1 cm×2 cm. The urethra 108 is supported by a hammock-like structure formed of the anterior vaginal wall suspended from the levator muscles, and the fascial attachments to the tendinous arch of the pelvic fascia. Those skilled in the art will understand that the size and location of the treatment regions 106 is preferably selected to achieve the desired effect on those supports (i.e., to achieve a desired amount of lift of the urethra 108).

During or following the heat treatment of the endopelvic fascia with the high intensity ultrasound energy, a collagen substance may be inserted into the treatment site to further support the tightened tissue. For example, the collagen may be injected underneath the treatment regions 106 to act as a bulking agent to fill in areas surrounding the endopelvic fascia 114 that are or will be vacated as this tissue shrinks. In a different embodiment, collagen injected into the treatment area may also be subject to a second exposure to high energy ultrasound to stiffen the additional collagen and reduce an amount of dissipation and absorption which would otherwise be expected after a collagen injection. For example, as shown in FIG. 1, the secondary collagen 110 may be injected adjacent to the treatment regions 106, or at another location in the vicinity of the urethra 108 to form a more secure support for the urethra 108.

According to a different embodiment according to the invention, the collagen 110 is injected into the operative area before applying the ultrasonic heating treatment. When the heating is applied, both the collagen naturally present in the endopelvic fascia 114 and the additional collagen 110 harden with the injected collagen providing permanent support to the fascia 114 and further enhancing the correct positioning of the urethra 108 and bladder neck. In yet another embodiment, the injected collagen described above is combined with a binding protein selected to bind the injected material to the endopelvic fascia 114. Using a binding protein also increases the surface area heated during ultrasound energy delivery. This alternative procedure may be used successfully to treat surgical sites where the natural collagen content of the target tissue is insufficient to provide adequate shrinkage.

The exemplary procedures to treat incontinence described above make use of the tightening properties of collagen after it is heated. However, other embodiments may be devised that utilize a different agent adapted to bind with the endopelvic fascia 114 and cause its shortening. For example, shrinking agents that respond to heating by tightening or foreshortening may be applied to the fascia 114 before it is heated, as described above, using the high energy ultrasound probe according to the invention. These agents may be naturally occurring, or more typically may be injected in place to bind with the tissue to be treated before application of the ultrasound energy.

In addition to the treatment of stress urinary incontinence, the embodiments of the present invention may also be used to treat other conditions. For example, fecal incontinence may be successfully treated, by inserting into the rectum a probe similar to the high intensity ultrasound probe 102 and treating a region of the endopelvic fascia 114. As described above with reference to urinary incontinence, the specific region of the fascia 114 to be treated is preferably selected to maximize the resulting tightening of the fascia 114, and the ensuing reduction of the symptoms of incontinence.

A system according to the present invention may also be used during cosmetic procedures. For example, selected portions of collagen injected into the lips of a patient may be heated at intensities and for durations selected to sculpt the injected collagen into a desired shape. High intensity ultrasound may be used without collagen injection to heat collagen molecules in the skin and cause shrinkage. This technique may also be used in other applications, for example, removing varicose veins through heating.

The present invention has been described with reference to specific exemplary embodiments. Those skilled in the art will understand that changes may be made in details, particularly in matters of shape, size, material and arrangement of parts. Accordingly, various modifications and changes may be made to the embodiments, such as using the procedures described to shrink and tighten different types of biological tissues. The specifications and drawings are, therefore, to be regarded in an illustrative rather than a restrictive sense.

What is claimed is:

1. A method for treating incontinence, comprising:
inserting a high energy ultrasound probe into a body canal; and
transmitting ultrasound energy from the probe through a wall of the body canal to focus at a first treatment region of an adjacent portion of endopelvic fascia, an intensity and duration of the transmitted energy being controlled to heat the first treatment region sufficiently to obtain a desired shrinkage of a portion of the endopelvic fascia including the first treatment region.

2. The method according to claim 1, further comprising focusing the ultrasound energy on the first treatment region for between 2 minutes and 3 minutes.

3. The method according to claim 1, further comprising focusing the energy on a second treatment region at an intensity and for a duration controlled to heat the second treatment region sufficiently to obtain a desired shrinkage of a portion of the endopelvic fascia including the second treatment region.

4. The method according to claim 1, wherein the first treatment region has dimensions of approximately 1 cm by 2 cm.

5. The method according to claim 1, wherein the first treatment region is located approximately 1 cm lateral to the urethra.

6. The method according to claim 1, further comprising injecting collagen adjacent to the first treatment region to fill a void created by the shrinking of the portion of the endopelvic fascia including the first treatment region.

7. The method according to claim 7, further comprising focusing ultrasound energy on the injected collagen.

8. The method according to claim 1, further comprising injecting collagen into the first treatment region prior to heating the first treatment region.

9. The method according to claim 1, further comprising injecting into the first treatment region a non-collagen shrinking agent to bind to the treatment region.

10. The method according to claim 1, wherein the focused ultrasound energy heats the treatment region to a temperature of between about 85° C. and about 100° C.

11. The method according to claim 3, wherein the body canal is a vaginal canal and the treatment is for urinary incontinence.

12. The method according to claim 11, wherein the first and second treatment regions are located substantially symmetrically with respect to the urethra.
